(19) United States
(12) Patent Application Publication
(10) Pub. No.: US 2020/0199531 A1
(43) Pub. Date: Jun. 25, 2020

(54) 3D SUSPENSION METHOD FOR GENERATING AUTOLOGOUS MELANOCYTE BY INDUCING IPS CELLS AND APPLICATION THEREOF

(71) Applicant: JIANGSU UNIVERSITY, Jiangsu (CN)

(72) Inventors: Liping LIU, Jiangsu (CN); Yumei LI, Jiangsu (CN); Yunwen ZHENG, Jiangsu (CN); Ningning GUO, Jiangsu (CN)

(21) Appl. No.: 16/613,054

(22) PCT Filed: Jun. 28, 2019
(86) PCT No.: PCT/CN2019/093509

§ 371 Date: Nov. 12, 2019

(30) Foreign Application Priority Data
May 4, 2018 (CN) 201810419157.6

(51) Int. Cl. C12N 5/071; C12N 5/00

(52) U.S. Cl. CPC C12N 5/0626; C12N 5/0062; C12N 2506/45; C12N 2501/33; C12N 2533/52; C12N 2500/34; C12N 2513/00

(57) ABSTRACT

The present invention relates to the field of biological technology and relates to a method for growing cells, specifically relating to a 3D suspension method for growing autologous melanocyte by inducing iPS cells, and to an application thereof. Said method of the present invention detaches the iPS cells into single cells and uses 3D culture plates to grow embryoid bodies, which all have uniform shapes and sizes. The early term induction process 14 days before the differentiation replaces 2D planar monolayer cultivation with 3D suspension cultivation, thereby lowering the rate of epithelioid cell occurrences during the differentiation process, enhancing the differentiation efficiency of melanocytes, optimizing the pre-differentiation embryoid body selection, single cell detachment time, and culture medium components, and improving the proliferation state of melanocyte. The melanocyte obtained by means of the present invention has the characteristics of being highly similar to normal melanocyte in vitro and exhibits features markedly superior to normal melanocyte during in vivo transplantation.

### TECHNICAL FIELD

The present invention relates to a method for generating cells, and in particular relates to a method for inducing iPS cells to generate autologous melanocytes by using a three-dimensional suspension system and application thereof and belongs to the technical field of biology.

### BACKGROUND

Melanocytes are the only cell type producing melanin that protects skin cells from ultraviolet ray. Human melanocytes can be isolated directly from the epidermis; however, the very limited quantity of melanocytes in epidermis and their poor proliferation capability in vitro have limited the application of melanocytes in autologous cellular transplantation therapy and drug screening, etc.

Except for the epidermis, melanocytes can be obtained by differentiation through other ways, such as melanocyte stem cells and melanoblasts, dermal stem cells, hair follicle stem cells, stem cells of hair follicle outer root sheath, embryonic neural crest stem cells, and embryonic stem cells. However, these methods have disadvantages respectively. For example, the number of melanocyte stem cells, dermal stem cells and hair follicle-related stem cells in the skin is extremely low and they do not have infinite proliferation capability. As a result, the number of induced melanocytes is very limited. Although embryonic stem cells can proliferate infinitely, they have ethic issues and cannot be used to obtain autologous melanocyte for patient transplantation therapy.

Besides, skin fibroblasts and keratinocytes can be also directly transdifferentiated to obtain functional melanocytes by reprogramming method. However, the efficiency of the existing trans-differentiation systems is extremely low, and there is still a certain gap in function between the obtained cells and normal melanocytes, so the application is limited.

Induced pluripotent stem cells (iPS cells), which have unlimited proliferation and multi-directional differentiation capacities, can be differentiated into functional melanocytes in vitro by using specific factors according to several studies. Compared with other sources, iPS cells have many advantages: 1) autologous iPS cells can be established for the patient by minimally invasive or even non-invasive sampling and then differentiated into autologous melanocytes; 2) iPS cells propagate infinitely, so they could generate enough melanocytes for the patient; 3) there are no ethical issues; 4) genetic characteristics of patient can be maintained, and personalized therapy may be achieved when they are applied in pathogenesis studying and drug screening for the specific patient.

When the conventional scheme for iPS cells differentiation into melanocytes is used, large quantities of epithelium-like cells can be found during melanocytes generation. These epithelium-like cells are high in proportion and they also proliferate quickly, affecting the proliferation of melanocytes. Therefore, it is difficult to obtain mature melanocytes after several rounds of passage. There are two main reasons for the low-efficiency: 1) the size and morphology of embryoid bodies generated by using conventional methods such as mechanical dissociation method or tryptic dissociation method varies greatly; 2) the whole differentiation process of the conventional method is conducted in 2D flat system, easily resulting in mass epithelium-like cells with rapid proliferation capability.

### CONTENT OF THE INVENTION

The current differentiation schemes for melanocyte have the technical problems including: the various size and morphology of embryoid bodies, low differentiation efficiency, the differentiation being accompanied with generation of a large amount of epithelium-like cells, and difficulty in obtaining mature melanocytes after several rounds of passage. The invention aims to overcome these defects in the prior art and provides a novel method for generating autologous melanocyte, said method using a 3D suspension system to induce iPS cells to generate autologous melanocyte.

In order to achieve the above aim, the technical solution adopted by the invention is as follows:

a. Embryoid Body Formation by Using Single Cell Method

iPS single cell dissociation enzyme is added into iPS clones for dissociation; mTeSR medium is added and cells are pipetted gently to form iPS single cell suspension; after centrifugation, the supernatant is discarded, then mTeSR medium is added to cell pellet for resuspension; iPS single cells are counted and inoculated into three dimensional (3D) culture plate; ROCK inhibitor is added; after 24 hours of culture, embryo bodies having uniform morphology and size are obtained; the embryo bodies are aspirated by gentle pipette and transferred to a low attachment plate for continued suspension culture, and the medium is changed every day.

In step a, the iPS single cell dissociation enzyme is ACCUTASE™, the 3D culture plate is Elplasia™ 3D plate (24-well) and the inoculation density is 5×10⁵ cells per well, ROCK inhibitor is Y-27632, the suspension culture lasts for 5-10 days, and finally the embryoid bodies reach 300-500 μm in diameter.

b. 3D Suspension Differentiation:

(1) 3D early-stage differentiation:
The embryoid bodies in the low attachment plate for continued suspension culture which are obtained in step a are transferred into differentiation medium for early-stage differentiation, and the embryoid bodies are suspended in the low attachment plate during the early-stage differentiation.

(2) Mid-stage attached differentiation:
After the early-stage differentiation in the above step b(1), the embryoid bodies are transferred to a fibronectin-coated culture plate for mid-stage attached differentiation, the differentiation medium components remain unchanged, and the embryoid bodies attach on the plate and grow.

(3) Late-stage differentiation:
After attached culture in the above step b (2), the embryoid bodies are dissociated with enzyme into single cells, inoculated into a fibronectin-coated culture plate, and subjected to late-stage maturation induction in the optimized differentiation medium. When the cell density reaches 90%, passage is performed with dissociation enzymes, and mature melanocytes are obtained after 35 to 42 days of differentiation.

The early-stage differentiation described in step b (1) lasts for 14 days and mid-stage attached differentiation in step b(2) lasts for 7 days.

The embryoid bodies after attached culture described in step b (3) refer to embryoid bodies on day 21 of differentiation and the inoculation density is 2×10⁴/cm².

The optimized differentiation medium described in step b (3) includes: 50% (v/v) L-Wnt3a cell conditioned medium, 30% (v/v) low-glucose DMEM, 20% MCDB 201 medium, 0.05 μM dexamethasone, 1× insulin-transferrin-selenium, 1 mg/ml linoleic acid-bovine serum albumin, 10⁻⁴ M L-ascorbic acid, 50 ng/ml stem cell factor, 100 nM EDN3, 20 pM cholera toxin, 4 ng/ml basic fibroblast growth factor (bFGF) and 0.5% fetal bovine serum (FBS).

Compared to the prior art, the present invention has the following beneficial effects:

In the conventional 2D flat differentiation. system for melanocytes, a large amount of epithelium-like cells are found and the proportion of dendritic cells is extremely small. After single-cell dissociation, the flat and polygonal epithelium-like cells are still in high proportion and they proliferate quickly while the dendritic cells are in low proportion and proliferate slowly. As a result, mature melanocytes cannot be obtained frequently after several rounds of passage. According to the method disclosed by the invention, the early-stage differentiation process is conducted in 3D suspension culture system instead of 2D flat system. Large quantities of dendritic cells can be found in the periphery of embryoid bodies after attachment and the epithelium-like cells are rarely observed. These dendritic cells have a rapid proliferation capability in the optimized late-stage differentiation medium, and a large amount of mature melanocytes can be obtained after several rounds of passage.

Melanocytes generated by using the preparation method disclosed by the invention have excellent performance advantages. The melanocytes obtained in the present invention show high similarity to normal human melanocytes in terms of in vitro characteristics and their in vivo function is remarkably superior to that of normal melanocytes when transplanted into the skin of immunodeficient mice. Therefore, they are more beneficial to the application in patient autologous cell transplantation for treatment of depigmentation diseases such as vitiligo. Meanwhile, they are also more appropriate for the in vitro establishment of 3D skin model which can be used for patient pathogenesis studying and personalized drug screening.

### BRIEF DESCRIPTION OF DRAWINGS

FIG. 1 is a graph showing melanocyte differentiation by using the conventional 2D flat attached method. A shows morphology of embryoid bodies which are generated by using a traditional mechanical method; B shows morphology of embryoid bodies which have been transferred to the fibronectin-coated culture plate and are on day 21 of differentiation; C shows cellular morphology after single-cell dissociation performed on day 21 of differentiation, and D shows the cellular morphology after two rounds of passage. Scale: A, 500 μm; B-D, 200 μm.

FIG. 2 is a graph showing the induction of melanocytes from iPS cells by using 3D suspension system. A shows the morphology of iPS single cells inoculated into 3D culture plate; B shows the morphology of embryoid bodies which are formed 24 hours after adding Rock inhibitor; C shows the morphology of embryoid bodies which have been cultured for 7 days; D shows morphology of on day 14 of differentiation in the 3D suspension system; Ea shows the morphology on day 21 of differentiation (day 7 after attachment) and Eb is the local amplification graph of Ea; F shows morphology of cells on day 28 of differentiation and G shows morphology of melanocyte on day 35 of differentiation. Scale: Ea 500 μm; others: 200 μm.

FIG. 3 is a comparison diagram showing 3D suspension differentiation by using embryoid bodies with different culture days and sizes. A shows cellular morphology of embryoid bodies which are cultured for less than 3 days and have a diameter of less than 200 μm, B and C show the morphology of embryoid bodies shown in A on day 14 and 21 of differentiation, respectively; D shows embryoid bodies which are cultured for more than 14 days and have a diameter of more than 700 μm, E and F show the morphology of embryoid bodies shown in D on day 15 and 21 of differentiation, respectively. Scale: 200 μm.

FIG. 4 is a comparison graph showing the melanocyte proliferation conditions, wherein the melanocytes are obtained by single cell dissociation of the induced embryoid bodies at different time points. Scale: 200 μm:

FIG. 5 is a comparison graph showing the effect of different concentration of serum or serum substitute, which is added into the late-stage differentiation medium, on the proliferation of induced melanocyte. Scale: 200 μm.

FIG. 6 shows the identification graph of in vitro characteristics of iPS cells-derived melanocytes which are generated by using 3D suspension method. A indicates the mRNA expression levels of melanocyte characteristic genes (relative to housekeeping gene GAPDH); B shows the expression of melanocyte characteristic protein MITF-M, TYR and TYRPI; C is DOPA staining for the identification of tyrosinase activity; D is Masson-Fontana staining for the identification of melanin production. E shows melanosome generation detected by transmission electron microscope. Scale: B, 200 μm; C-D, 50 μm; E, 0.5 μm.

FIG. 7 is a comparison graph of the transplantation outcome of iPS cells-derived melanocytes and normal melanocytes in a mouse hair follicle reconstitution assay. Scale in M-F staining: 200 μm.

FIG. 8 shows the graph of differentiation of different iPS cell lines using 3D suspension system. A shows the induction process of cell line iPSCs-2; Aa shows cell line iPSCs-2 under normal culture condition and Ab shows embryoid bodies generated from iPSCs-2 cell line; Ac shows day 14 of differentiation, Ad shows day 21 of differentiation, Ae shows day 28 of differentiation and Af shows day 35 of differentiation; B shows the induction process of cell line iPSCs-3; Ba shows iPSCs-3 cell under normal culture condition and Bb shows embryoid bodies generated from iPSCs-3 cell line; Be shows day 14 of differentiation, Bd shows day 21 of differentiation and Be is the local amplification graph of Bd; Bf shows day 35 of differentiation. Scale: Bd, 500 μm; others, 100 μm.

FIG. 9 shows the identification graph of iPS cells which are generated by reprogramming of human skin fibroblasts using virus transfection method. A shows human skin fibroblasts and B shows established iPS cells; C shows alkaline phosphatase staining; D shows the mRNA expression level of stemness genes; E shows structure of three germ layers in teratoma formed from iPS cells. Scale: A-C, 200 μm; E, 100 μm.

### EMBODIMENTS

The present invention is further described below with reference to the examples, but it should not be understood that the subject scope of the present invention is limited only to the following examples. Various substitutions and modifications can be made according to ordinary technical knowledge and conventional means in the art without departing from the technical ideas of the present invention, all of which should be included within the protection scope of the present invention. The experimental methods used in the examples which have no special instructions are all conventional methods. The materials, reagents and the like used in the following examples without special instructions can be obtained from a commercial route.

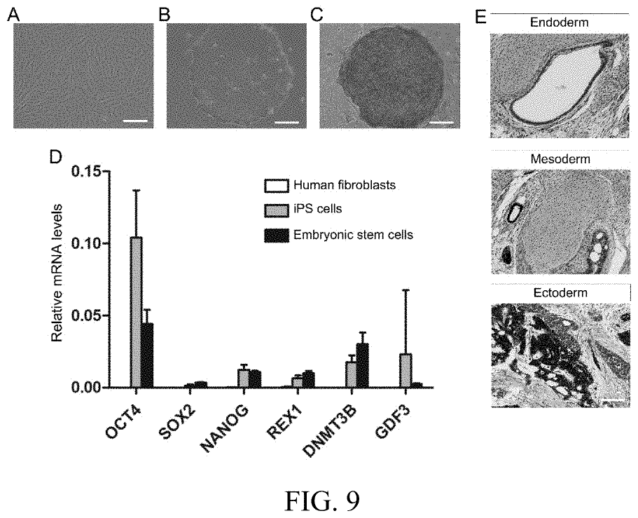

In the present invention, iPS cells, iPSCs-2 cells and iPSCs-3 cells are generated from human skin fibroblasts by virus transfection method and the characteristics of generated iPS cells are identified as shown in FIG. 9. A shows human skin fibroblasts and B shows established iPS cells; C shows alkaline phosphatase staining; D shows the mRNA expression level of stemness genes (relative to housekeeping gene GAPDH), OCT4, SOX2, NANOG, REX1, DNMT3B and GDF3 in the horizontal axis are stemness characteristic genes and their expression levels in iPS cells and in embryonic stem cells are at a comparable level, while they cannot be detected in human fibroblasts. E shows the structure of three germ layers in teratoma formed from iPS cells.

### Example 1

Conventional Method (2D Differentiation):

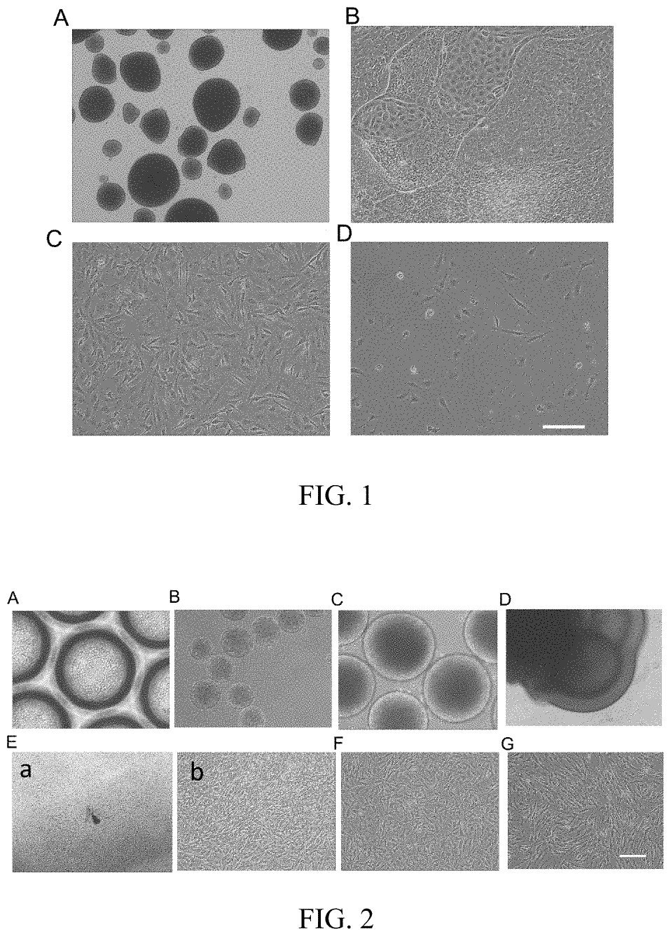

As shown in FIG. 1, embryoid bodies produced by using the conventional method (such as mechanical method) are various in terms of size and morphology (FIG. 1A); these embryoid bodies will entirely spread out during 2D flat culture, and a large quantities of epithelium-like cells will be generated while the proportion of dendritic cells is very small (FIG. 1B); after single cell dissociation on day 21 of differentiation, it can be observed that in the attached single cells, the majority of cells maintain a morphology of polygonal epithelium-like cell, while the proportion of dendritic cells is low (FIG. 1C); It is usually difficult to obtain mature melanocytes after a couple of passage due to the low proportion and slow proliferation rate of melanocytes as well as the high proportion and quick proliferation of epithelium-like cells (FIG. 1D).

### Example 2

a. Embryoid Body Formation by Using Single Cell Method

When iPS cell clones grow to a suitable size, add iPS single cell dissociation enzyme ACCUTASE™ (Innovative Cell Technologies). Place for 5-7 min at room temperature. Add mTeSR (Stemcell Technologies) medium and gently pipette to form iPS single cell suspension. Centrifuge and discard the supernatant. Add mTeSR medium to resuspend and count. Inoculate iPS single cells into Elplasia™ 3D culture plate (Kuraray) (FIG. 2A). Taking a 24-well plate as an example for inoculation density, add 5×10⁵ cells into each well and add ROCK inhibitor, Y-27632 (Wako) to a final concentration of 10 M. Culture in a 37° C. incubator. After 24 h, embryoid bodies with uniform size and morphology are generated (FIG. 2B). Gently pipette the embryoid bodies and transfer them into the low attachment plate (Corning) for further culture. Change the medium every day.

b. 3D Suspension Differentiation Process

(1) 3D Early-Stage Differentiation:

When embryoid bodies reach 300-500 μm in diameter after being cultured for 5-10 days in low attachment plate (FIG. 2C), they are transferred into differentiation medium to start early-stage differentiation. It is still conducted in low attachment plate and it lasts for 14 days. It can be observed that embryoid bodies enlarge gradually with their color being darkened and their morphology becoming irregular (FIG. 2D).

The differentiation medium includes: 50% (v/v) L Wnt-3a cell conditioned medium, 30% low-glucose DMEM (Gibco), 20% MCDB 201 (Sigma-Aldrich), 0.05 μM dexamethasone (Sigma-Aldrich), lx insulin-transferrin-selenium (Sigma-Aldrich), 1 mg/ml linoleic acid-bovine serum albumin (Sigma-Aldrich), 10⁻⁴ M L-ascorbic acid (Sigma-Aldrich), 50 ng/ml stem cell factor (Sigma-Aldrich), 100 nM EDN3 (American Peptide Company), 20 pM cholera toxin (Sigma-Aldrich), 50 nM 12-O-tetradecanoyl-phorbol-13-acetate (TPA) (Sigma-Aldrich) and 4 ng/mL bFGF (Wako).

(2) Mid-Stage Attached Differentiation

Preparation of fibronectin (BD Biosciences)-coated culture plate: for each well of a 6-well plate, add 1 ml DPBS and 20 μl fibronectin stock-solution (1 mg/ml). Pipette gently for homogeneous mixing. Incubate at room temperature for 1 h. Discard and wash once with 1 ml DPBS for the next step.

Transfer embryoid bodies which have been differentiated for 14 days in step (1) to above mentioned fibronectin-coated culture plates for mid-stage attached differentiation with the components of differentiation medium remaining unchanged. Attached embryoid bodies grow for another 7 days. At this time, a great number of dendritic cells are generated in the peripheral area of embryoid bodies and epithelium-like cells are rarely found (FIG. 2Ea, 2Eb). These dendritic cells maintain the ability of rapid proliferation.

(3) Late-Stage Differentiation

Dissociate the embryoid bodies after attached differentiation in Step (2) into single cells using TrypLE Select (Invitrogen) and inoculate them onto fibronectin-coated culture plate (on day 21 of differentiation). The inoculation density is 2×10⁴/cm². Late-stage differentiation is performed in the optimized differentiation medium. When the cell density reached 90%, the cells are dissociated using TrypLE Select and passaged. The dendrites become more and more typical and these cells proliferate quickly (FIG. 2F). Mature melanocytes are obtained on days 35-42 of differentiation (FIG. 2G).

The optimized differentiation medium includes: 50% (v/v) L-Wnt3a cell conditioned medium, 30% low-glucose DMEM medium, 20% MCDB 201 medium, 0.05 μM dexamethasone, lx insulin-transferrin-selenium, 1 mg/ml linoleic acid-bovine serum albumin, 10⁻⁴ M L-ascorbic acid, 50 ng/ml stem cell factor, 100 nM EDN3, 20 pM cholera toxin, 4 ng/ml bFGF and 0.5% FBS.

### Example 3

3D Suspension Differentiation by Using Embryoid Bodies with Different Culture Days and Sizes:

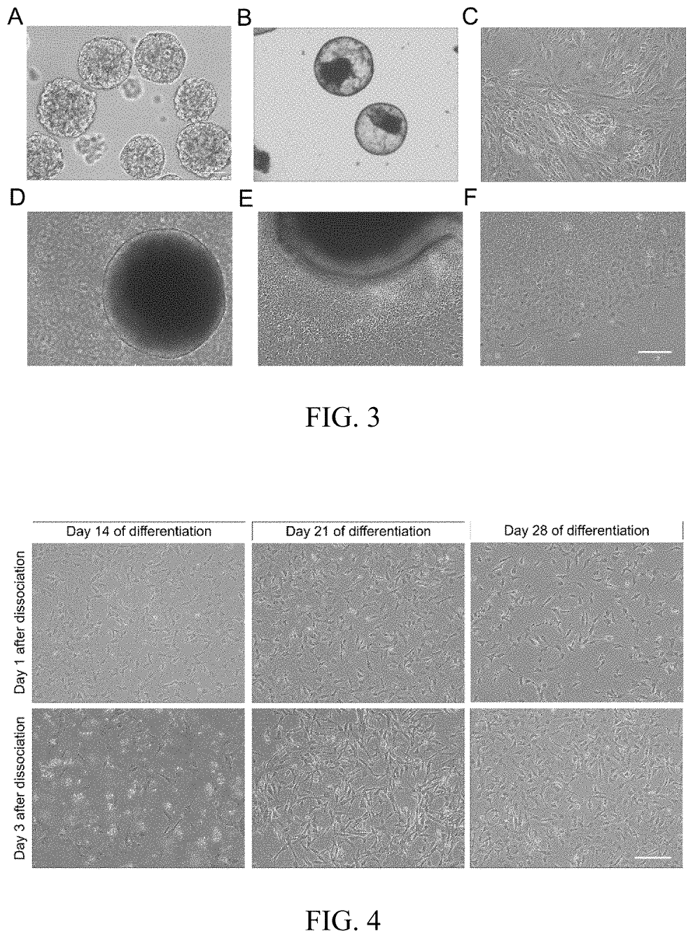

The effect of different culture days and sizes of embryoid bodies on melanocyte differentiation is studied in the present invention. As shown in FIG. 3, in the embryoid bodies which have been cultured for less than 3 days and have a diameter of less than 200 μM (FIG. 3A), many cavities appear in the early stage of differentiation (on day 14) (FIG. 3B). They show a flat morphology and have no proliferation capability after attachment (on day 21 of differentiation) (FIG. 3C). In the embryoid bodies which have been cultured for more than 14 days and have a diameter of more than 700 pun (FIG. 3D), most of the cells in the central dark region could not migrate to form the single cell and fail to proliferate after attachment (on day 15 of differentiation) (FIG. 3E). The peripheral cells also show epithelium-like morphology on day 21 of differentiation of the embryoid bodies (FIG. 3F).

### Example 4

Effect of Different Time Points of Single Cell Dissociation on Melanocyte Differentiation:

In the process of single cell dissociation of the embryoid bodies after attached culture in Example 2, the present invention compared the effects of different time points of single cell dissociation on the proliferation state of induced melanocytes. Single cell dissociation and passage of embryoid bodies are conducted on day 14, 21 and 28 of differentiation. As shown in FIG. 4, cells that have undergone dissociation on the 14th and 28th day of differentiation cannot proliferate or proliferate slowly. By contrast, cells that have undergone dissociation on day 21 of differentiation still keep a high proliferation activity and can continue to differentiate into mature melanocytes which are more similar to normal melanocytes in morphology. Therefore, it is finally determined that single cell dissociation is best carried out on day 21 of differentiation, which is more beneficial to melanocytes growth.

### Example 5

Effect of the Serum with Different Concentrations on Melanocyte Proliferation:

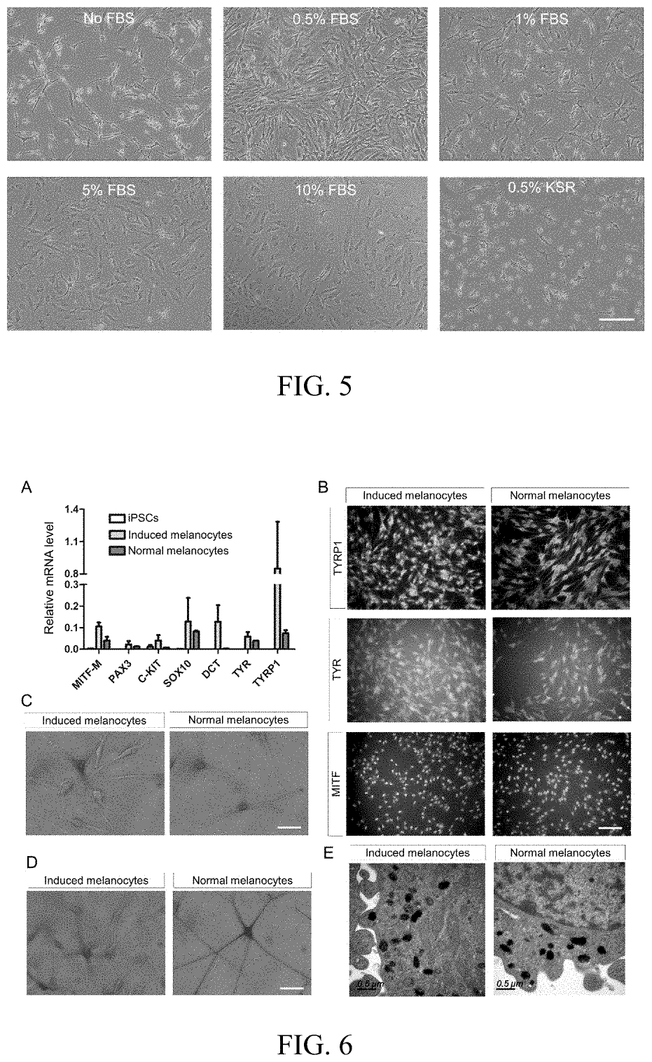

In the culture process after single cell dissociation according to Example 2, the effects of adding serum FBS (Gibco) and knockout serum replacement (KSR, Gibco) with different concentrations in the differentiation medium on melanocyte growth are compared in the present invention. As shown in FIG. 5, melanocytes without the addition of serum (no FBS) have a weak prolificacy, while addition of high-concentration FBS (1%, 5% and 10%) lead to premature senescence of melanocytes. By contrast, addition of 0.5% FBS improves melanocyte proliferation remarkably, however, using 0.5% serum replacement KSR cannot significantly improve its proliferation status. Therefore, addition of 0.5% serum is determined to be optimal for improving the proliferation status of melanocyte.

### Example 6

Identification of In Vitro Characteristics of Autologous Melanocytes Generated by Inducing iPS Cells Using 3D Suspension System:

The in vitro characteristic comparison is performed between the induced melanocytes prepared by the method according to the present invention and the normal melanocyte, as shown in FIG. 6. In FIG. 6A, MITF-M, PAX3, c-KIT, SOX10, DCT, TYR and TYRPI in the horizontal axis are melanocyte characteristic genes and their expression levels in the induced melanocytes are equivalent to those in the normal melanocytes, while they cannot be detected in iPS cells. As shown in FIG. 6B, the expression of the melanocyte characteristic proteins MITF-M, TYR and TYRPI in the induced melanocytes are close to those in normal melanocytes. As shown in FIGS. 6C and 6D, DOPA-staining for tyrosinase activity identification and Masson-Fontana staining for melanogenesis identification show positive results in both cells. As shown in FIG. 6E, mature melanosome generation is detected in both induced melanocytes and normal melanocytes by transmission electron microscopy. Therefore, the induced melanocytes obtained according to the present invention and human normal melanocytes have highly similar in vitro characteristics.

### Example 7

Identification of the In Vivo Function of the Induced Melanocytes Generated by 3D Suspension System:

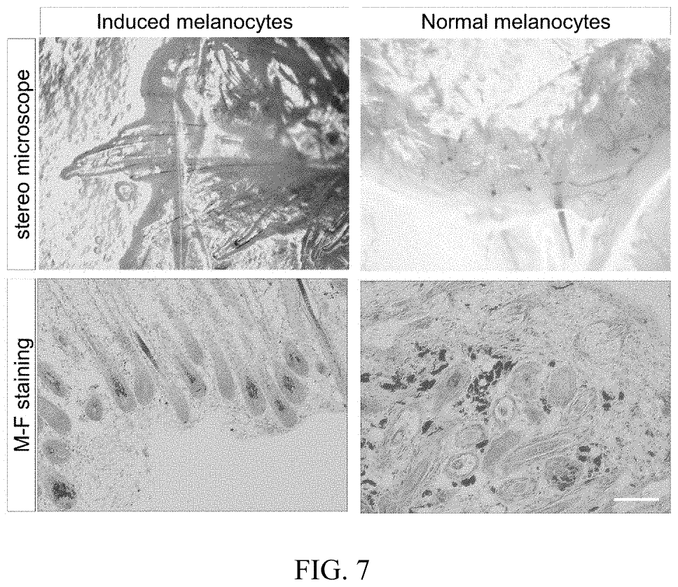

The in vivo function comparison is performed between the induced melanocytes prepared according to the preparation method of the present invention and normal melanocytes. As shown in FIG. 7, when the induced melanocytes obtained according to the present invention are transplanted into the skin of immunodeficient mice using a hair follicle reconstitution assay, black hair follicles and long hair shafts can be found under stereo microscope. Masson-Fontana (M-F) staining also shows melanin localized in the hair bulb and hair shaft, suggesting that these cells have the ability to transfer melanin to surrounding keratinocytes. By contrast, only short black hair follicles were observed under stereo microscope when human normal melanocytes have been transplanted and there is no long black shaft. M-F staining showed only a small number of melanocytes can be integrated into the hair bulb, and the majority of cells die and leave massive melanin deposits in the surrounding connective tissue.

Therefore, induced melanocytes obtained in this invention are obviously superior to normal melanocytes in terms of in vivo function, and they are more beneficial to future transplantation application.

### Example 8

Application of 3D Suspension System on Different iPS Cell Lines:

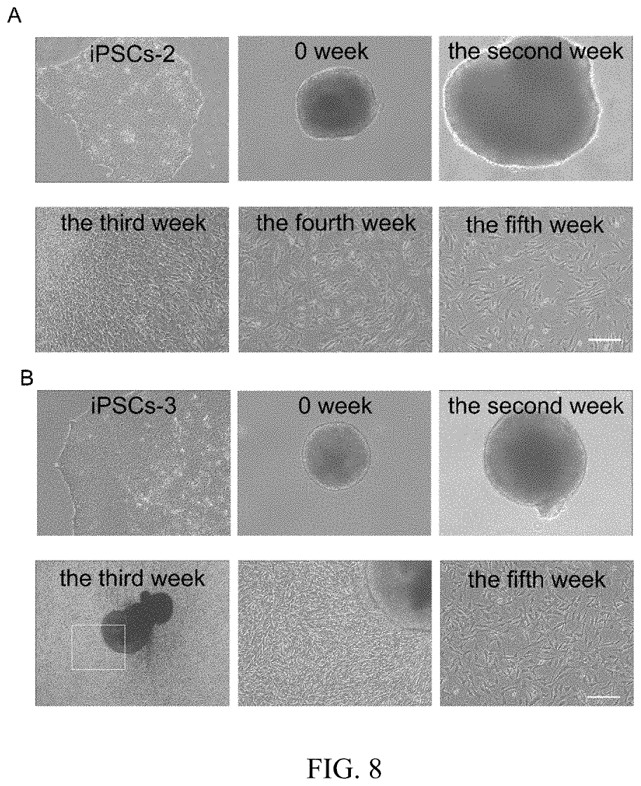

The 3D suspension system for in vitro inducing iPS cells to generate autologous melanocytes was applied to the other two iPS cell lines: iPSCs-2 cells and iPSCs-3 cells, and a large number of mature melanocytes were efficiently induced, suggesting that the method has wide applicability. As shown in FIGS. 8A and 8B, large quantities of mature melanocytes were obtained by inducing the iPSCs-2 cell line and iPSCs-3 cell line.

1. A method of inducing iPS cells to generate autologous melanocytes by using 3D suspension system, characterized in that, said method comprises the following steps:
a) embryoid body formation by using single cell method
iPS single cell dissociation enzyme is added into iPS clones for dissociation; mTeSR medium is added and cells are pipetted gently to form iPS single cell suspension; after centrifugation, the supernatant is discarded, then mTeSR medium is added to cell pellet for resuspension; iPS single cells are counted and inoculated into three dimensional culture plate; ROCK inhibitor is added; after culture, embryo bodies having uniform morphology and size are obtained; the embryo bodies are aspirated by gentle pipette and transferred to a low-attachment plate for continued culture, and the medium is changed every day; and
b) 3D suspension differentiation:
(1) 3D early-stage differentiation: the embryoid bodies obtained in step a) are transferred into differentiation medium for early-stage differentiation,
(2) mid-stage attached differentiation: after the early-stage differentiation in the above step b)(1), the embryoid bodies are transferred to a fibronectin-coated culture plate for mid-stage attached culture, the differentiation medium components remain unchanged, and the embryoid bodies attach to the plate and grow, and
(3) late-stage differentiation: after attached culture in the above step b)(2), the embryoid bodies are dissociated into single cells, inoculated into a fibronectin-coated culture plate, and subjected to late maturation induction in differentiation medium, when the cell density reaches 90%, passage is performed with dissociation enzymes, and mature melanocytes are obtained after 35 to 42 days of differentiation.

2. The method for generating autologous melanocytes according to claim 1, characterized in that, the three dimensional culture plate for iPS single cells inoculation is Elplasia™ three dimensional plate (24 wells) and the inoculation density is 5×10⁵ cells per well.

3. The method for generating autologous melanocytes according to claim 1, characterized in that, the continued culture lasts for 5-10 days until embryoid bodies reach 300-500 μm in diameter.

4. The method for generating autologous melanocytes according to claim 1, characterized in that, the embryoid bodies in step b) (1) are suspended in the low-attachment plates during the early-stage differentiation.

5. The method for generating autologous melanocyte according to claim 1, characterized in that, the early-stage differentiation in step b) (1) lasts for 14 days and mid-stage attached differentiation in step b) (2) lasts for 7 days.

6. The method for generating autologous melanocyte according to claim 1, characterized in that, the embryoid bodies after attached culture in step b) (3) are embryoid bodies on day 21 of differentiation.

7. The method for generating autologous melanocyte according to claim 1, characterized in that, the density of inoculation in step b) (3) is 2×10⁴/cm².

8. The method for generating autologous melanocyte according to claim 1, characterized in that, the differentiation medium described in step b) (3) includes: 50% (v/v) L-Wnt3a cell conditioned medium, 30% low-glucose DMEM, 20% MCDB 201 medium, 0.05 μM dexamethasone, lx insulin-transferrin-selenium, 1 mg/ml linoleic acid-bovine serum albumin, 10⁻⁴ M L-ascorbic acid, 50 ng/ml stem cell factor, 100 nM EDN3, 20 pM cholera toxin, 4 ng/ml bFGF and 0.5% fetal bovine serum.

9. The use of a melanocyte prepared according to the method of claim 1, characterized in that, the melanocyte is used for cellular transplantation or drug screening for treatment of depigmented diseases.

10. The use according to claim 9, characterized in that, the depigmented diseases is vitiligo.
